(19) United States
(12) Patent Application Publication
(10) Pub. No.: US 2014/0150800 A1
(43) Pub. Date: Jun. 5, 2014

(54) PATIENT INTERFACE ARRANGEMENT WITH ADJUSTABLE FOREHEAD SUPPORT

(71) Applicant: KONINKLIJKE PHILIPS ELECTRONICS N.V., Eindhoven (NL)

(72) Inventors: Harmina Christina ZEIJLSTRA, BREDA (NL); Sander Theodoor PASTOOR, UTRECHT (NL)

(73) Assignee: KONINKLIJKE PHILIPS ELECTRONICS N.V., Eindhoven (NL)

(21) Appl. No.: 13/692,114

(22) Filed: Dec. 3, 2012

(51) Int. Cl. A61M 16/06

(52) U.S. Cl. CPC A61M 16/0683
USPC 128/207.11; 128/207.13

(57) ABSTRACT

The invention provides a patient interface arrangement which has a forehead support which is adjustable by a rotary knob. A sound damping means is provided for damping sounds generated when there is rotational adjustment or clattering of the adjustable components.

The present invention relates to patient interfaces for transporting a gas to and/or from an airway of a user.

There are numerous situations where it is necessary or desirable to deliver a flow of breathing gas non-invasively to the airway of a patient, i.e. without inserting a tube into the airway of the patient or surgically inserting a tracheal tube in their oesophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnoea syndrome, in particular, obstructive sleep apnoea (OSA).

Non-invasive ventilation and pressure support therapies involve the placement of a patient interface device including a mask component on the face of a patient. The mask component may be, without limitation, a nasal mask that covers the patient's nose, a nasal pillow/cushion having nasal prongs that are received within the patient's nostrils, a nasal/oral mask that covers the nose and mouth, or a full face mask that covers the patient's face. The patient interface device interfaces between the ventilator or pressure support device and the airway of the patient, so that a flow of breathing gas can be delivered from the pressure/flow generating device to the airway of the patient.

Such devices are typically maintained on the face of a patient by headgear having one or more straps adapted to fit over/around the patient's head.

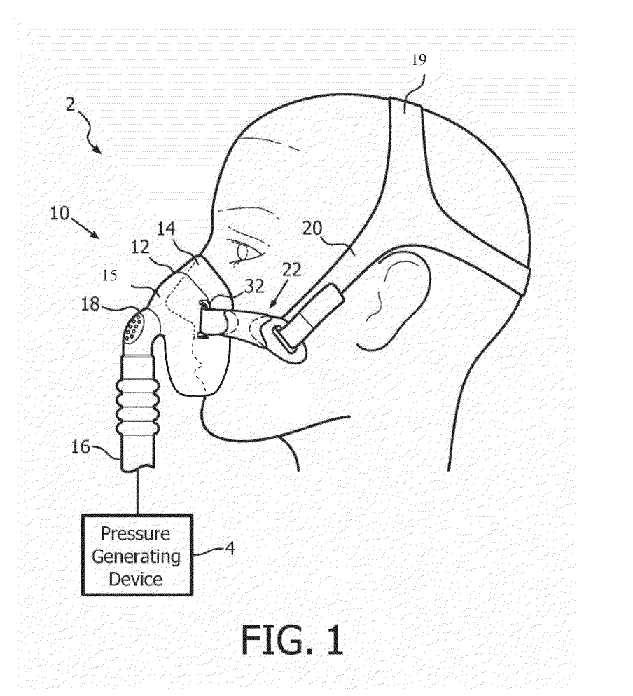

FIG. 1 shows a typical system to provide respiratory therapy to a patient. This system will be referred to in the description and claims as a “patient interface arrangement”.

The system 2 includes a pressure generating device 4, a delivery conduit 16 coupled to an elbow connector 18, and a patient interface device 10. The pressure generating device 4 is structured to generate a flow of breathing gas and may include, without limitation, ventilators, constant pressure support devices (such as a continuous positive airway pressure device, or CPAP device), variable pressure devices, and auto-titration pressure support devices.

Delivery conduit 16 communicates the flow of breathing gas from pressure generating device 4 to patient interface device 10 through the elbow connector 18. The delivery conduit 16, elbow connector 18 and patient interface device 10 are often collectively referred to as a patient circuit.

The patient interface device 10 includes a mask 12, which in the exemplary embodiment is nasal and oral mask. However, any type of mask, such as a nasal-only mask, a nasal pillow/cushion or a full face mask, which facilitates the delivery of the flow of breathing gas to the airway of a patient, may be used as mask 12. The mask 12 includes a cushion 14 coupled to a shell 15. The cushion 14 is made of a soft, flexible material, such as, without limitation, silicone, an appropriately soft thermoplastic elastomer, a closed cell foam, or any combination of such materials. An opening in shell 15, to which elbow connector 18 is coupled, allows the flow of breathing gas from pressure generating device 4 to be communicated to an interior space defined by the shell 15 and cushion 14, and then to the airway of a patient.

The patient interface device 10 also includes a headgear component 19, which in the illustrated embodiment is a two-point headgear. Headgear component 19 includes a first and a second strap 20, each of which is structured to be positioned on the side of the face of the patient above the patient's ear.

Headgear component 19 further includes a first and a second mask attachment element 22 to couple the end of one of the straps 20 to the respective side of mask 12.

A problem with this type of mask is that the headgear force vectors necessary to achieve a robust and stable seal against the face of the patient can cut a straight line near the corners of a patient's eyes, which can be uncomfortable and distracting.

In order to avoid this, it is well known to include a forehead support to spread the required forces over a larger area. In this way, an additional cushion support on the forehead balances the forces put by the mask around the nose or nose and mouth.

In order to allow the mask to be a comfortable fit for a variety of users, the forehead support needs to be adjustable with respect to the nasal/oral mask part. In one known design, this is achieved by providing the forehead support on the end of an arm which is pivotally connected to the main nasal/oral mask part. This enables the forehead support to be pivoted towards and away from the forehead, to allow different relative positioning for different users.

Another known design has a forehead support that is mounted for linear movement between retracted and extended positions. A rotary adjustment knob is used to adjust the linear position of the forehead support, by means of a threaded interface.

This known design is described in detail in US 2010/0000542 which is hereby incorporated by reference.

A problem with this design is that the threaded interface causes noises, both during adjustment and when the adjustment is complete. A clattering noise is caused by the accuracy of the engagement between threaded shafts, and a clicking noise is caused by a ratchet type engagement which gives the rotary knob pre-set positions. As a result of the close proximity to the wearer's face (and therefore the ears), these noises can be disturbing.

Furthermore, with one possible use of the mask for treating sleep apnoea, such noises are particularly undesirable.

According to the invention, there is provided a patient interface arrangement as claimed in claim 1. The arrangement has a forehead support with an adjustment mechanism in the form of a rotary knob having a threaded drive shaft, and a threaded driven shaft. Rotation of the rotary knob causes linear movement of the driven shaft and thereby also the support cushion. A sound damping means is provided for damping sounds generated when there is relative movement of the drive shaft and driven shaft.

This arrangement makes the adjustment and/or use of the patient interface quieter so that less discomfort is caused to the patient.

The drive shaft can comprise a central core and a surrounding thread (i.e. a screw), and the driven shaft then comprises an outer cylindrical shaft and an inner thread (i.e. a threaded bore).

In a first arrangement, the sound damping means comprises an elastic coating on the threaded (external) drive shaft. In a second arrangement, the sound damping means comprises an elastic coating on the threaded (internal) driven shaft. In both cases, this reduces noise when the shafts are rotated relative to each other (i.e. during adjustment) and it also assists in maintaining the two shafts at a fixed relative position (thereby avoiding clattering) when the adjustment has been completed. The elastic coating can be a rubber coating.

In a third arrangement, the sound damping means comprises a least one additional thread on the threaded drive shaft and at least one corresponding additional thread on the threaded driven shaft, such that the drive shaft and the driven shaft are coupled by at least two threads in parallel. This provides a stronger hold between the shafts, to prevent relative movement after adjustment and to limit play during adjustment, and thereby reduce rattling noise.

In a fourth arrangement, the sound damping means comprises a resilient member for linearly biasing the drive shaft relative to the driven shaft. This again reduces rattling by maintaining a more fixed relative position.

The resilient member can be a spring mounted within the outer cylindrical shaft against the end of the central core. In this way, the spring is internally mounted within the threaded shaft arrangement. Alternatively, the resilient member can be a spring or rubber component around the base of a handle of the rotary knob.

The rotary knob can comprise rest positions defined by the interaction between a tab and a recess, wherein there is a plurality of tabs and/or a plurality of recesses to define a plurality of rest positions of the rotary knob, wherein the tab or tabs are coated with an elastic material.

This coating makes adjustment clicks between pre-set rotary knob positions quieter. The recess or recesses can instead (or as well) be coated with an elastic material.

Examples of the invention will now be described in detail with reference to the accompanying drawings, in which:

FIG. 1 shows a known patient interface;

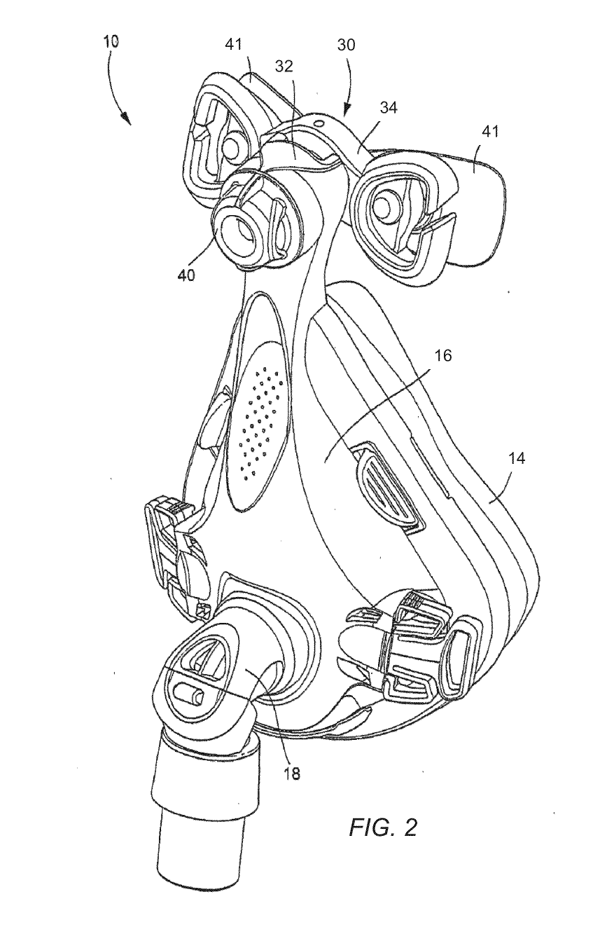

FIG. 2 shows a known patient interface as disclosed in US2010/0000542;

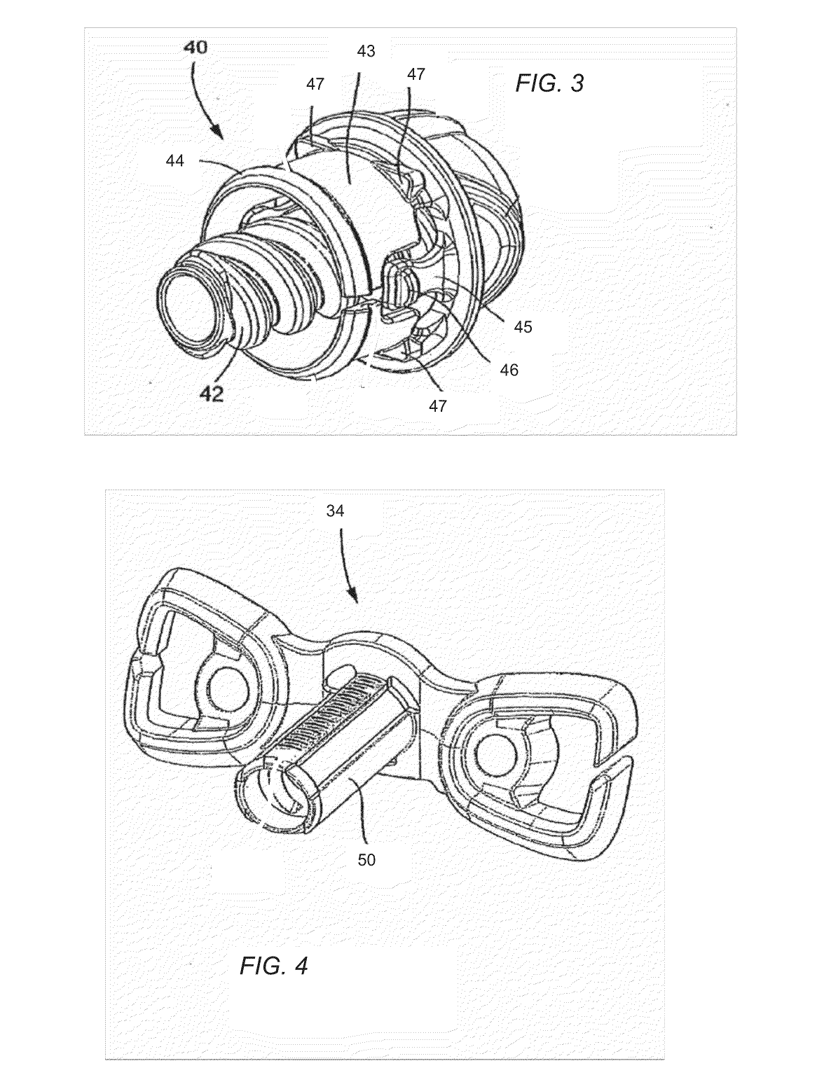

FIG. 3 shows the rotary knob of the interface of FIG. 2 in more detail;

FIG. 4 shows the forehead support of the interface of FIG. 2 in more detail;

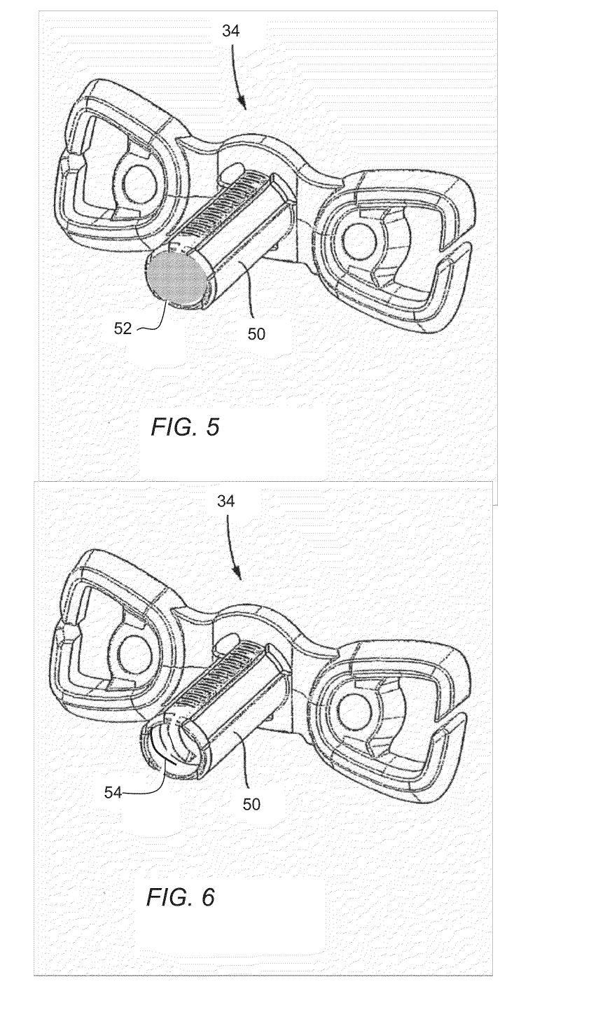

FIG. 5 shows a first way of providing sound damping in accordance with the invention;

FIG. 6 shows a second way of providing sound damping in accordance with the invention;

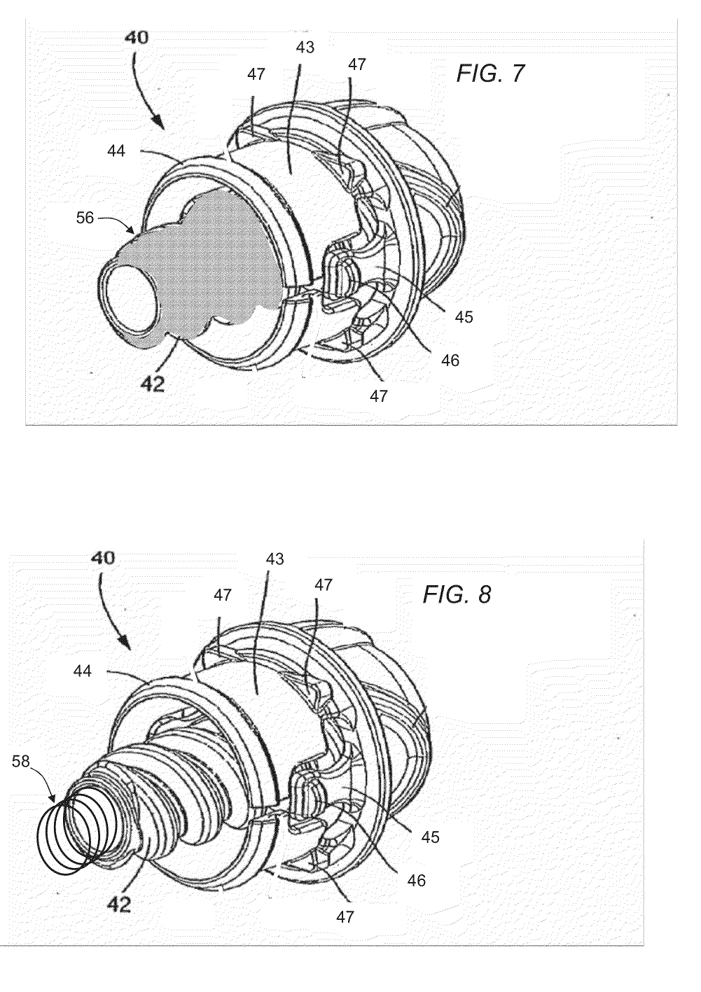

FIG. 7 shows a third way of providing sound damping in accordance with the invention;

FIG. 8 shows a fourth way of providing sound damping in accordance with the invention;

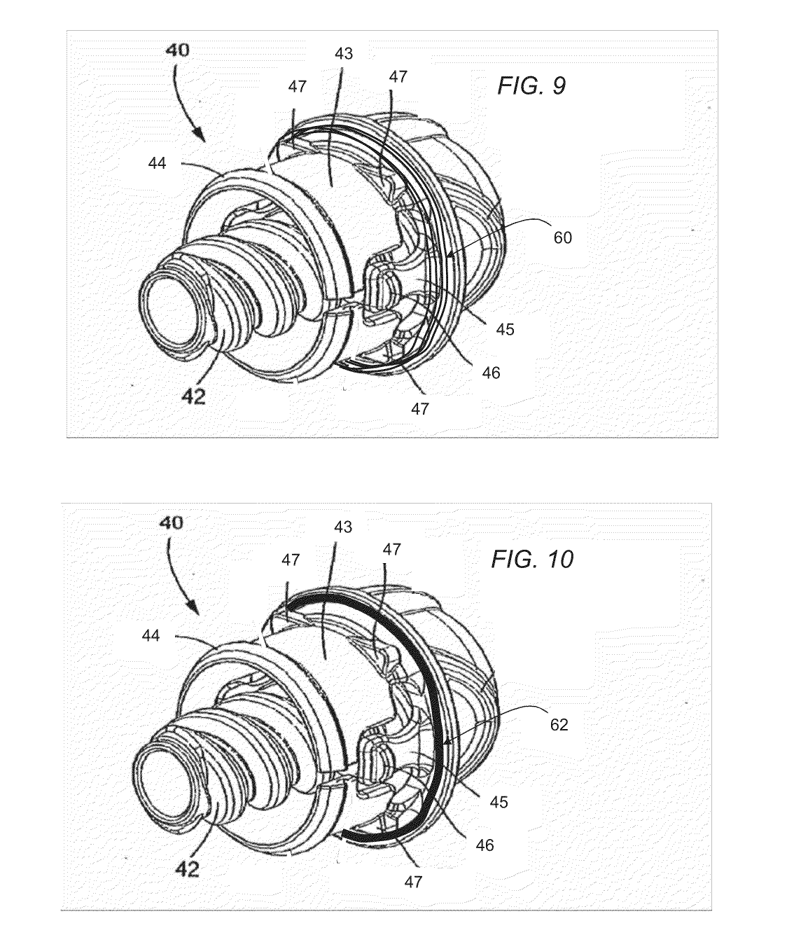

FIG. 9 shows a fifth way of providing sound damping in accordance with the invention;

FIG. 10 shows a sixth way of providing sound damping in accordance with the invention;

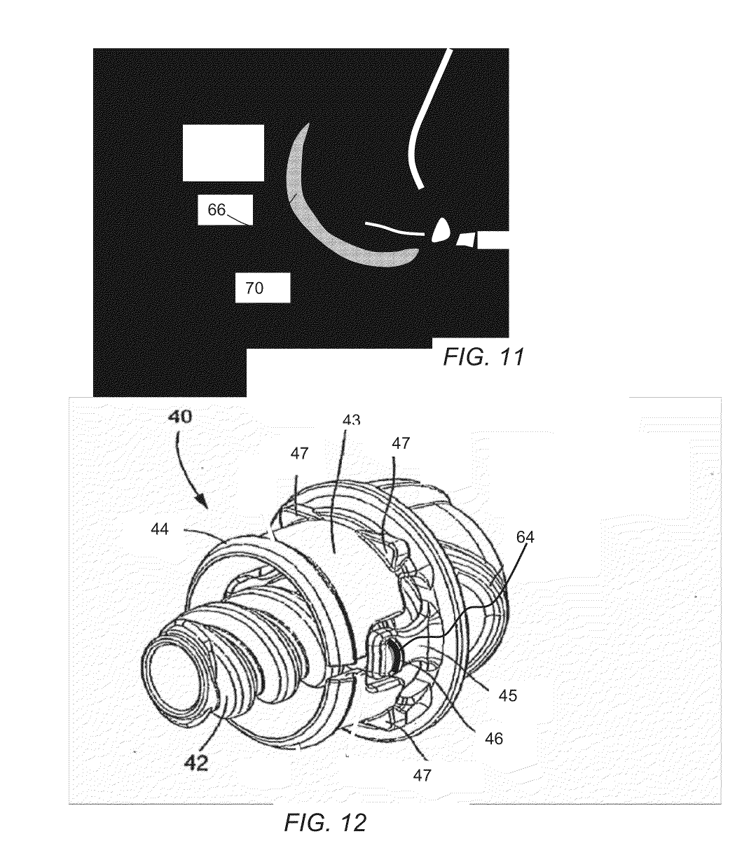

FIG. 11 shows a seventh way of providing sound damping in accordance with the invention; and

FIG. 12 shows an eighth way of providing sound damping in accordance with the invention.

The invention provides a patient interface arrangement which has a forehead support which is adjustable by a rotary knob. Sound damping is provided for damping sounds generated when there is rotational adjustment or clattering of the adjustable components.

FIG. 2 is taken from US2010/0000542 and shows a patient interface arrangement in the form of a full facial mask assembly 10 including a forehead support 30. The patient interface device for delivering gas to the user (the mask part) includes a frame 16, a cushion 14 adapted to form a seal with the patient's face, an elbow assembly 18 for connection to an air delivery tube (components 10,14,16,18 corresponding to those of the same number in FIG. 1).

The forehead support 30 uses a screw-type actuator to move the forehead support 30 along a generally linear path. The main components of the forehead support 30 are a frame connector 32 connected to the mask frame 16, a rotary adjustment knob 40 including a threaded shaft, and a forehead cushion support 34 including an internally threaded tube. The forehead cushion support 34 carries the forehead cushions 41 that engage the patient's forehead in use.

When the adjustment knob 40 is rotated, the internally threaded tube of the forehead cushion support 34 extends or retracts from the threaded shaft of the adjustment knob 40, which causes adjustable movement of the forehead cushions 41.

FIG. 3 shows the rotary knob 40 in more detail and shows the threaded shaft 42. The rotary knob 40 clips onto the frame connector 32 with a snap-fit. Specifically, the knob 40 includes a segment 43 that is inserted into the frame connector 20. An annular rim 44 is provided on the segment 43 that can be engaged with retention features provided in the frame connector 32. The threaded shaft 42 is adapted to engage within an internally threaded tube of the forehead cushion support 34 such that the threaded shaft 42 is engaged with the internally threaded tube. When the knob 40 is rotated, the internally threaded tube extends or retracts from the threaded shaft 42 which causes adjustable movement of the forehead cushions 41.

A resilient tab 45 including a ratchet bump 46 is located on each side of the adjustment knob 40. When the knob 40 is assembled to the frame connector 32, the ratchet bumps 46 engage a series of ridges around the interior surface of the frame connector to provide a series of ratchet positions. Centring features 47 are provided to make initial fitting of the rotary knob 40 easier.

FIG. 4 shows the forehead cushion support 34 having a tube 50 with an internal thread, for receiving the threaded shaft of the rotary knob 40.

Thus, the rotary knob has a drive shaft comprising a central core and a surrounding thread 42, and the driven shaft of the forehead support 34 comprises an outer cylindrical shaft 50 and an inner thread.

The components have only been described to the extent required to understand the improvements of this invention. Full details of the system can be found in US 2010/0000542.

FIG. 5 shows a first way of providing sound damping in accordance with the invention.

The forehead support 34 is provided with rubber coating 52 inside the central threaded bore. The coating prevents that when the rotary knob 40 is screwed into the forehead support, there can be a clattering noise. The rubber coating provides friction and thus a reduction of the noise. The plastic parts are no longer touching each other.

FIG. 6 shows a second way of providing sound damping in accordance with the invention.

The forehead support 34 is provided with an additional internal thread or threads 54. The outer threads of the rotary knob 40 correspond, so that there are multiple threads in parallel. By having more threads, the threaded shaft of the rotary knob 40 is supported during the movement and makes less clattering noise.

FIG. 7 shows a third way of providing sound damping in accordance with the invention.

The outer thread 42 on the rotary knob 40 has a rubber coating 56. This coating prevents that when the rotary knob is screwed into the forehead support 34 there is a clattering noise.

FIG. 8 shows a fourth way of providing sound damping in accordance with the invention.

The back of the tube 50 of the forehead support 34 is closed and a spring 58 is positioned in the forehead support 34. This spring extends between the end of the threaded shaft of the rotary knob 40 and the closed end of the tube 50.

This spring provides a bias which reduces the clattering noise.

FIG. 9 shows a fifth way of providing sound damping in accordance with the invention. A spring 60 is positioned around the rotary knob, just behind the handle. When the rotary knob is clicked into position into the frame connector 32, the spring 60 makes sure that the rotation knob is better positioned and provides a bias to limit axial movement.

FIG. 10 shows a sixth way of providing sound damping in accordance with the invention.

Instead of the spring 60, a rubber ring 62 is positioned around the rotary knob 40, for the same purpose as the spring 60. Instead, the rubber ring 62 may be implemented as a surface coating applied to the required part of the rotary knob, so that the component count remains the same.

FIGS. 11 and 12 shows a seventh and eighth way of providing sound damping in accordance with the invention.

As shown in FIG. 11, the frame connector 32 can have a series of ridges and troughs 70 that engage the ratchet bumps 46 provided on the knob 40 (as shown in FIG. 3). These provide rest positions defined by the interaction between tabs (the ratchet bumps 46) and recesses of the pattern 70. There is a plurality of tabs and/or a plurality of recesses to define a plurality of rest positions of the rotary knob.

FIG. 11 shows the seventh approach of providing a rubber coating 66 in the ridge and recess pattern 70 to prevent the ratchet bump 46 making noise when it is in contact with the ridge and trough pattern 70 in the frame connector 32.

FIG. 12 shows the eighth approach of providing the ratchet bump 46 of the rotation knob 40 with a rubber coating 64, again to prevent the ratchet bump 46 making noise when it is in contact with the ridge and trough pattern 70 in the frame connector 32.

The various measures described above can be combined in any combination to achieve improved performance. The invention improves the user comfort by minimising avoidable noises.

The invention has been described as modifications to one specific design. However, the invention can be applied to any rotary adjustable forehead support, and is based on the recognition that the screw thread interaction within the rotary adjustment mechanism provides noise problems.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface arrangement comprising:
a patient interface device for communicating with the nose or the nose and mouth of a patient; and
a forehead support coupled to the patient interface device and having a support cushion;
wherein the forehead support comprises an adjustment mechanism which comprises a rotary knob having a threaded drive shaft, and a threaded driven shaft coupled to the rotary knob drive shaft and to the support cushion, wherein rotation of the rotary knob causes linear movement of the driven shaft and thereby also the support cushion,
wherein the forehead support further comprises a sound damping means for damping sounds generated when there is relative movement of the drive shaft and driven shaft.

2. An arrangement as claimed in claim 1, further comprising a strap arrangement for holding the patient interface device and forehead support against the head of the patient,

3. An arrangement as claimed in claim 1, wherein the drive shaft comprises a central core and a surrounding thread, and the driven shaft comprises an outer cylindrical shaft and an inner thread.

4. An arrangement as claimed in claim 1, wherein the sound damping means comprises an elastic coating on the threaded drive shaft.

5. An arrangement as claimed in claim 4, wherein the sound damping means comprises a rubber coating.

6. An arrangement as claimed in claim 1, wherein the sound damping means comprises an elastic coating on the threaded driven shaft.

7. An arrangement as claimed in claim 6, wherein the sound damping means comprises a rubber coating.

8. An arrangement as claimed in claim 1, wherein the sound damping means comprises a least one additional thread on the threaded drive shaft and at least one corresponding additional thread on the threaded driven shaft, such that the drive shaft and the driven shaft are coupled by at least two threads in parallel.

9. An arrangement as claimed in claim 1, wherein the sound damping means comprises a resilient member for linearly biasing the drive shaft relative to the driven shaft.

10. An arrangement as claimed in claim 9, wherein the drive shaft comprises a central core and a surrounding thread, and the driven shaft comprises an outer cylindrical shaft and an inner thread, and wherein the resilient member is mounted within the outer cylindrical shaft against the end of the central core.

11. An arrangement as claimed in claim 10, wherein the resilient member comprises a spring.

12. An arrangement as claimed in claim 1, wherein the sound damping means comprises a resilient member for linearly positioning the drive shaft relative to the driven shaft, mounted around the base of a handle of the rotary knob.

13. An arrangement as claimed in claim 12, wherein the resilient member comprises a spring or a rubber component.

14. An arrangement as claimed in claim 1, wherein the rotary knob comprises rest positions defined by the interaction between a tab and a recess, wherein there is a plurality of tabs and/or a plurality of recesses to define a plurality of rest positions of the rotary knob, wherein the tab or tabs are coated with an elastic material.

15. An arrangement as claimed in claim 1, wherein the rotary knob comprises rest positions defined by the interaction between a tab and a recess, wherein there is a plurality of tabs and/or a plurality of recesses to define a plurality of rest positions of the rotary knob, wherein the recess or recesses are coated with an elastic material.
